(19) United States
(12) Patent Application Publication
(10) Pub. No.: US 2021/0053039 A1
(43) Pub. Date: Feb. 25, 2021

(54) CATALYST FOR PRODUCING UNSATURATED CARBOXYLIC ACID

(71) Applicant: NIPPON KAYAKU KABUSHIKI KAISHA, Tokyo (JP)

(72) Inventors: Toru KAWAGUCHI, Yamaguchi (JP); Seiichiro FUKUNAGA, Yamaguchi (JP); Bungo NISHIZAWA, Yamaguchi (JP); Tomohiro OBATA, Yamaguchi (JP); Yasuhiro ARATANI, Yamaguchi (JP); Hideomi SAKAI, Yamaguchi (JP); Tomoyuki EJIRI, Yamaguchi (JP)

(73) Assignee: NIPPON KAYAKU KABUSHIKI KAISHA, Tokyo (JP)

(21) Appl. No.: 17/040,374

(22) PCT Filed: Mar. 25, 2019
(86) PCT No.: PCT/JP2019/012445

§ 371 Date: Sep. 22, 2020

(30) Foreign Application Priority Data
Mar. 28, 2018 (JP) 2018-062652

(51) Int. Cl. B01J 27/199; C07C 51/235; C07C 51/25; B01J 37/00; B01J 37/08; B01J 6/00

(52) U.S. Cl. CPC B01J 27/199; C07C 51/235; B01J 6/001; B01J 37/0045; B01J 37/08; C07C 51/25

(57) ABSTRACT

Provided is a catalyst for producing an unsaturated carboxylic acid, in which a ratio of a diffraction line intensity of 2θ=19.1±0.3° with respect to a diffraction line intensity of 2θ=10.7±0.3° in X-ray diffraction measurement is 0.20 or more and less than 0.58, and the catalyst having an active component represented by formula (A) shown below:

Mo₁₀VₐP_{b}Cu_{c}As_{d}XₑO_{g} (A)

### TECHNICAL FIELD

The present invention relates to a highly selective catalyst for producing an unsaturated carboxylic acid.

### BACKGROUND ART

A number of catalysts have been proposed as catalysts used to produce unsaturated carboxylic acids. A catalyst for producing methacrylic acid among unsaturated carboxylic acids is mainly composed of molybdenum and phosphorus, and has a structure of heteropolyacid and/or a salt thereof. A number of methods for producing these catalysts have also been proposed.

It is described in Non-Patent Document 1 that the heteropolyacid catalyst, which is not a partially neutralized salt, changes the structure of the Keggin anion due to heat treatment and constitution water loss.

Many proposals have been made as to catalysts for producing methacrylic acid. In Patent Document 1, a method has been proposed in which a catalyst for producing methacrylic acid is obtained with good productivity without accompanying performance degradation by controlling the % by mass of the organic substance in the catalyst precursor of the heteropolyacid partially neutralized salt, the weight reduction rate at the time of temperature rise, and the rate of temperature rise.

In Patent Document 2, a method for producing a catalyst for producing methacrylic acid has been proposed in which the catalyst precursor of the heteropolyacid partially neutralized salt is heat-treated at least twice at a temperature of 350 to 500° C. for 1 to 30 hours under a gas flow, the catalyst precursor is once cooled to below 250° C. between the each heat treatments, and the difference in heat treatment temperature of each time is within 30° C.

In Patent Document 3, a method for producing a catalyst for producing methacrylic acid has been proposed in which when molding is performed by a coating method using Mo—V—P—Cu heteropolyacid as an active component and using water or alcohol and/or an aqueous solution of alcohol as a binder, the water content of the catalyst powder used for the molding, the temperature and humidity of the molding process, and the temperature and humidity of the calcination process are controlled.

As to these known techniques, in Patent Document 1 the heteropolyacid partially neutralized salt is used and there is a concern about the service life when used industrially for a long period of time. In Patent Document 2, since the two-step calcination process is performed, it is not economical and there is concern about a stable method for producing a catalyst. Further, in Patent Document 3 further improvement in the yield of methacrylic acid is required. Moreover, the catalysts obtained by the method of Patent Document 1 to Patent Document 3 are not yet sufficient in the reaction performance, and further improvement is desired at the time of use as an industrial catalyst.

### PRIOR ART DOCUMENTS

### Patent Document

Patent Document 1: Japanese Patent No. 5485013
Patent Document 2: Japanese Patent No. 3581038
Patent Document 3: Japanese Patent No. 5570142

### Non-Patent Document

Non-Patent Document 1: Applied Catalysis A: General 210 (2001), 13-34

### SUMMARY OF THE INVENTION

### Problems to be Solved by the Invention

An object of the present invention is to provide a catalyst capable of stably producing an unsaturated carboxylic acid with excellent selectivity.

### Means for Solving the Problems

As a result of intensive investigations to solve the problems described above, the inventors have found that a catalyst, which has a ratio of a diffraction line intensity of 2θ=19.1±0.3° with respect to a diffraction line intensity of 2θ=10.7±0.3° in X-ray diffraction measurement of 0.20 or more and less than 0.58, has high unsaturated carboxylic acid selectivity to complete the invention.

That is, the present invention relates to:

A catalyst for producing an unsaturated carboxylic acid, wherein a ratio of a diffraction line intensity of 2θ=19.1±0.3° with respect to a diffraction line intensity of 2θ=10.7±0.3° in X-ray diffraction measurement is 0.20 or more and less than 0.58, and the catalyst comprising an active component represented by formula (A) shown below:

Mo₁₀VₐP_{b}Cu_{c}As_{d}XₑO_{g} (A)

(in the formula, Mo, V, P, Cu, As and O represent molybdenum, vanadium, phosphorus, copper, arsenic and oxygen, respectively, and X represents at least one element selected from the group consisting of Ag, Mg, Zn, Al, B, Ge, Sn, Pb, Ti, Zr, Sb, Cr, Re, Bi, W, Fe, Co, Ni, Ce and Th; a, b, c, d, e and g each represents an atomic ratio of the element, and a satisfies 0.1≤a≤6, b satisfies 0.5≤b≤6, c satisfies 0<c≤3, d satisfies 0<d<3, e satisfies 0≤e≤3 and g is a value determined by the valence and atomic ratio of the other elements;)

The catalyst, wherein the unsaturated carboxylic acid is methacrylic acid; A method for producing the catalyst including the following steps:

(a) a step of mixing compounds containing an active component with water to prepare an aqueous solution or aqueous dispersion of these compounds,
(b) a step of drying the slurry solution obtained in the step (a) to obtain a dried slurry,
(c) a step of molding the dried slurry obtained in the step (b), and
(d) a step of calcining a molded product obtained in the step (c);

A method for producing an unsaturated carboxylic acid, wherein an unsaturated aldehyde is gas phase catalytic oxidized by molecular oxygen using the catalyst; and

The method, wherein the unsaturated aldehyde is methacrolein and the unsaturated carboxylic acid is methacrylic acid.

### Effects of the Invention

According to the present invention, it is possible to provide a highly selective catalyst having Mo, V, P, Cu, and As as essential elements and a method for producing the catalyst.

### BRIEF DESCRIPTION OF DRAWINGS

FIG. 1 is a diagram showing an X-ray diffraction spectrum of a catalyst of Example 1.

### EMBODIMENTS FOR CARRYING OUT THE INVENTION

The catalyst of the invention has a composition represented by the formula (formula A) shown below, and a ratio of a diffraction line intensity of 2θ=19.1±0.3° with respect to a diffraction line intensity of 2θ=10.7±0.3° in X-ray diffraction measurement of 0.20 or more and less than 0.58.

Mo₁₀VₐP_{b}Cu_{c}As_{d}XₑO_{g} (A)

(in the formula, Mo, V, P, Cu, As and O represent molybdenum, vanadium, phosphorus, copper, arsenic and oxygen, respectively, and X represents at least one element selected from the group consisting of Ag, Mg, Zn, Al, B, Ge, Sn, Pb, Ti, Zr, Sb, Cr, Re, Bi, W, Fe, Co, Ni, Ce and Th; a, b, c, d, e and g each represents an atomic ratio of the element, and a satisfies 0.1≤a≤6, b satisfies 0.5≤b≤6, c satisfies 0<c≤3, d satisfies 0<d<3, e satisfies 0≤e≤3 and g is a value determined by the valence and atomic ratio of the other elements.)

The active component of the catalyst of the invention may contain any active component element other than the essential active component elements of Mo (molybdenum), V (vanadium), P (phosphorus), Cu (copper) and As (arsenic), for example, Ag (silver), Mg (magnesium), Zn (zinc), Al (aluminum), B (boron), Ge (germanium), Sn (tin), Pb (lead), Ti (titanium), Zr (zirconium), Sb (antimony), Cr (chromium), Re (rhenium), Bi (bismuth), W (tungsten), Fe (iron), Co (cobalt), Ni (nickel), Ce (cerium) and Th (thorium).

The catalyst of the invention obtained by the production method of the invention is used in the production of an unsaturated carboxylic acid by gas phase catalytic oxidation of an unsaturated aldehyde with molecular oxygen, and in particular, it is suitably used in the production of methacrylic acid by gas phase contact oxidation of methacrolein with molecular oxygen.

The lower limit of a in the composition of formula (A) shown above is preferably 0.2, more preferably 0.3, and particularly preferably 0.4. The upper limit of a is preferably 4, more preferably 2, and particularly preferably 1. The lower limit of b in the composition of formula (A) shown above is preferably 0.6, more preferably 0.7, and particularly preferably 0.8. The upper limit of b is preferably 5, more preferably 4, and particularly preferably 2. The lower limit of c in the composition of formula (A) shown above is preferably 0.05, and more preferably 0.1. The upper limit of c is preferably 2, more preferably 1, and particularly preferably 0.3. The lower limit of d in the composition of formula (A) shown above is preferably 0.1, more preferably 0.2, and particularly preferably 0.3. The upper limit of d is preferably 2, more preferably 1.5, and particularly preferably 1. The upper limit of e in the composition of formula (A) shown above is preferably 2, more preferably 1.5, and particularly preferably 1.

The method for producing a catalyst of the invention includes (a) a step of dispersing compounds containing each or a plurality of the metals described above in water to prepare an aqueous solution or aqueous dispersion of these compounds (hereinafter collectively referred to as a slurry solution), (b) a step of drying the slurry solution obtained in step (a) to obtain a dried slurry, (c) a step of molding the dried slurry obtained in step (b), and (d) a step of calcinating a coated molded product obtained in step (c). Hereinafter, preferred embodiments of the respective steps are described, but the implementation of the invention is not limited to the embodiments described below.

The step (a) includes a step of preparing compounds containing an active component element and a step of mixing the compounds and water.

In the step (a), compounds containing an essential active component element and an optional active component element of the catalyst of the invention are used. Examples of the compound include a chloride, a sulfate, a nitrate, an oxide or an acetate of the active component element. Specific examples of preferred compound include nitrates, for example, cobalt nitrate, acetates, for example, copper acetate, oxides, for example, molybdenum oxide, vanadium pentoxide, copper oxide, antimony trioxide, cerium oxide, zinc oxide or germanium oxide, and an acid (or a salt thereof), for example, orthophosphoric acid, phosphoric acid, boric acid, aluminum phosphate or 12-tungstophosphoric acid (or salt thereof), but are not limited thereto. The compounds containing the active components may be used alone or in combination of two or more thereof. In the step (a), compounds containing each active component and water are mixed uniformly to obtain a slurry solution. In the slurry solution, it is not necessary that all the components are dissolved in water, and a part or the whole thereof may be in a suspended state. The amount of water used in the slurry solution is not particularly limited as long as the total amount of the compound used can be completely dissolved or mixed uniformly. The amount of water used may be appropriately determined in consideration of the drying method and drying conditions in the step (b). Usually, the amount of water is approximately from 200 to 2,000 parts with respect to 100 parts of the total mass of the slurry solution preparing compounds. The amount of water may be large, but if it is too large, the energy cost of the drying step in the step (b) increases, and many disadvantages, for example, the case where it cannot be completely dried may occur.

In the invention, the shape of the stirring blade of the stirrer used in the step (a) is not particularly limited, and any stirring blade, for example, a propeller blade, a turbine blade, a paddle blade, an inclined paddle blade, a screw blade, an anchor blade, a ribbon blade or a large lattice blade can be used in one stage, or the same blades or different blades can be used in two or more stages in the vertical direction. Further, a baffle (baffle plate) may be installed in a reaction tank, if desired.

In the step (b), the slurry solution obtained in the step (a) is completely dried. The drying method is not particularly limited, and includes, for example, drum drying, freeze drying, spray drying and evaporation to dryness. Among these, spray drying which can dry the slurry solution into powder or granules in a short time is preferred in the invention. The drying temperature for spray drying varies depending on the concentration of the slurry solution, the solution feeding speed, and the like, but the temperature at the outlet of the dryer is approximately from 70 to 150° C.

The step (c) includes a step of calcinating the dried slurry obtained in the step (b) (this step is not essential), a step of mixing the dried slurry with an additive, and a step of molding the dried slurry or a mixture of the dried slurry and the additive.

In the step (c), the dried slurry obtained in the step (b) is molded. When the dried slurry is calcinated at approximately from 250 to 350° C. and then molded, mechanical strength and catalyst performance may be improved in some cases so that the dried slurry may be calcinated before molding. The molding method is not particularly limited, and in order to reduce the pressure loss of the reaction gas in the oxidation reaction, the dried slurry is molded into a columnar shape, tablet, ring shape, spherical shape or the like, or an inert carrier may be coated with the dried slurry. Of these, since improvement in the selectivity and removal of reaction heat can be expected, it is preferred that the dried slurry is coated on an inert carrier to form a coated catalyst. This coating step is preferably the rolling granulation method described below. This method is, for example, a method, in a device having a flat or uneven disk at the bottom of a fixed container, of vigorously stirring the carrier in the container by means of repeated rotation motion and revolution motion by rotating the disk at high speed, and coating the carrier with a coating mixture including a binder and the dried slurry obtained in the step (b) or the coating mixture in which, if desired, other additive, for example, a molding auxiliary agent or a strength improver is added on the carrier. As to the method of adding the binder, any method, for example, of 1) preliminarily mixing with the coating mixture, 2) adding at the same time as adding the coating mixture to the fixed container, 3) adding after adding the coating mixture to the fixed container, 4) adding before adding the coating mixture to the fixed container, or 5) dividing the coating mixture and the binder and adding the total amounts thereof by an appropriate combination of 2) to 4) may be appropriately adopted. In 5), it is preferred to adjust the addition rate using an auto feeder or the like in order that a predetermined amount is carried on the carrier without adhesion of the coating mixture onto the fixed container, aggregation of the coating mixture or the like. The binder is preferably water or at least one selected from the group consisting of organic compounds having a boiling point of 150° C. or lower at 1 atm or lower, or an aqueous solution thereof. Specific examples of the binder other than water include an alcohol, for example, methanol, ethanol, propanols or butanols, preferably an alcohol having from 1 to 4 carbon atoms, an ether, for example, ethyl ether, butyl ether or dioxane, an ester, for example, ethyl acetate or butyl acetate, a ketone, for example, acetone or methyl ethyl ketone, and aqueous solutions thereof, and ethanol is particularly preferred. In the case of using ethanol as the binder, ethanol/water is 10/0 to 0/10 (mass ratio), preferably 9/1 to 1/9 (mass ratio) by mixing with water. The amount of the binder used is usually from 2 to 60 parts by weight, preferably from 10 to 50 parts by weight, based on 100 parts by weight of the coating mixture.

Specific examples of the carrier in the coating described above include a spherical carrier having a diameter of 1 to 15 mm, preferably 2.5 to 10 mm of silicon carbide, alumina, silica alumina, mullite, alundum and the like. The carrier used usually has a porosity of 10 to 70%. The ratio of the carrier to the coating mixture used is usually in an amount of coating mixture/(coating mixture+carrier) of 10 to 75% by mass, preferably 15 to 60% by mass. When the ratio of the coating mixture is large, the reaction activity of the coated catalyst increases, but the mechanical strength tends to decrease. On the contrary, when the ratio of the coating mixture is small, the mechanical strength is large, but the reaction activity tends to decrease. In the above, examples of the molding auxiliary agent used if desired include silica gel, diatomaceous earth and alumina powder. The amount of the molding auxiliary agent used is usually from 1 to 60 parts by mass with respect to 100 parts by mass of the catalytically active component solid. Further, if desired, it is useful to improve the mechanical strength of the catalyst to use an inorganic fiber (for example, ceramic fiber or whisker) inert to the catalytically active component and the reaction gas as a strength improver. The amount of the fiber used is usually from 1 to 30 parts by mass with respect to 100 parts by mass of the catalytically active component solid.

In the step (d), the molded dried slurry of the step (b) or the coated catalyst obtained in the step (c) is calcined. The dried slurry or the coated catalyst can be directly used as a catalyst for the catalytic gas phase oxidation reaction, but is preferably calcined because the structure is stabilized and the catalyst performance is improved when calcined. Further, if the calcination temperature is too high, the heteropolyacid may be decomposed and the catalyst performance may be deteriorated. Therefore, the calcination temperature is usually from 100 to 400° C., preferably from 250 to 380° C., more preferably from 320 to 380° C., and particularly preferably from 320 to 350° C. If the calcination time is too short, there is a concern that the structure of the heteropolyacid becomes unstable and the catalyst performance decreases, and if it is too long, the production efficiency of the catalyst decreases. The usual calcination time is from 1 to 20 hours. The calcination is usually performed in an air atmosphere, but may be performed in an inert gas atmosphere, for example, nitrogen or a reducing gas atmosphere, for example, ethanol. After the calcination in the inert gas or reducing gas atmosphere, if desired, calcination may be further performed in an air atmosphere. The ratio of the active component to the whole coated catalyst after the calcination obtained as described above is from 10 to 60% by mass.

The catalyst of the invention produced by the steps (a) to (d) described above has the diffraction line intensity of 2θ=19.1±0.3° with respect to the diffraction line intensity of 2θ=10.7±0.3° in the X-ray diffraction measurement of 0.20 or more and less than 0.58.

It is not preferred when the ratio is less than 0.20, the crystal structure of the heteropolyacid is decomposed and molybdenum oxide is generated, resulting in low activity and low selectivity, and when it is 0.58 or more, the selectivity decreases. The upper limit of the ratio is preferably 0.52 or less, and more preferably 0.50 or less.

The catalyst of the invention obtained by the method for producing a catalyst of the invention described above is used in a reaction for obtaining an unsaturated carboxylic acid by gas phase catalytic oxidation of an unsaturated aldehyde with molecular oxygen. Among them, it is suitably used for production of methacrylic acid by subjecting methacrolein to gas phase catalytic oxidation.

Molecular oxygen or molecular oxygen-containing gas is used for the gas phase catalytic oxidation reaction. The malar ratio of molecular oxygen to unsaturated aldehyde, for example, methacrolein used is preferably in the range of 0.5 to 20, and particularly preferably in the range of 1 to 10. For example, it is preferred to add water to the raw material gas in a molar ratio of 1 to 20 with respect to methacrolein for the purpose of allowing the reaction to proceed smoothly. The raw material gas may contain a gas inert to the reaction, for example, nitrogen, carbon dioxide, saturated hydrocarbon, and the like in addition to oxygen and, if desired, water (usually included as water vapor).

Further, as the unsaturated aldehyde used as a raw material, a gas containing an unsaturated aldehyde obtained by oxidizing an alkene compound, an alcohol compound or an ether compound as the raw material may be used as it is. In the case of methacrolein, a gas containing methacrolein obtained by oxidizing isobutylene, tertiary butanol and methyl tertiary butyl ether may be supplied as it is.

In the gas phase catalytic oxidation reaction, the reaction temperature is usually from 200 to 400° C., preferably from 260 to 360° C., the supply amount of the raw material gas in the space velocity (SV) is usually from 100 to 6,000 hr⁻¹, preferably from 300 to 3,000 hr⁻¹. The gas phase catalytic oxidation reaction can be performed under pressure or under reduced pressure, but ordinarily a pressure around atmospheric pressure is suitable.

The X-ray diffraction measurement of the catalyst of the invention is performed with the following apparatus and conditions.

Device used: (Ultima IV, manufactured by Rigaku Corporation)

### X-ray: CuKα ray (λ=0.154 nm)

### Output: 40 kV, 30 mA

Divergence slit: Open
Divergence vertical limit slit: 10 mm
Scattering slit; Open
Light receiving slit: 0.3 mm
Measurement range: 10 to 20°
Measurement speed: 10° per minute

### Step: 0.02°

Scan type: Continuous

Further, in the case of producing an unsaturated carboxylic acid using the catalyst of the invention, it is preferred to produce a corresponding unsaturated aldehyde using propylene, isobutylene, tert-butyl alcohol or the like as a raw material and a catalyst having an active component represented by formula (B) shown below.

Mo₁₂Bi_{a′}Fe_{b′}Co_{c′}Ni_{d′}X_{e′}Y_{f′}Z_{g′}O_{h′} (B)

(in the formula, X is at least one element selected from the group consisting of magnesium (Mg), calcium (Ca), manganese (Mn), copper (Cu), zinc (Zn), cerium (Ce) and samarium (Sm), Y is at least one element selected from the group consisting of boron (B), phosphorus (P), arsenic (As), antimony (Sb) and tungsten (W), and Z is at least one element selected from the group consisting of sodium (Na), potassium (K), rubidium (Rb) and cesium (Cs), a′ to g′ represents an atomic ratio of each component, h′ is a numerical value determined by an oxidation degree of a catalyst component, a′ is 0.40 or more and less than 0.80, b′ is 1.0 to 2.5, c′ is 4.5 to 7.5, d′ is 1.6 to 3.5, e′ is 0 to 10, f′ is 0 to 10, and g′ is 0.015 to 0.12.)

### EXAMPLE

Hereinafter, the invention will be described in more detail with reference to the examples, but the invention is not limited to the examples. In the examples, part means part by weight and % means % by weight, respectively.

### Example 1

### 1) Preparation of Catalyst

To 7,100 parts of pure water were added 1,000 parts of molybdenum oxide, 31.59 parts of vanadium pentoxide, 11.05 parts of cupric oxide, 80.07 parts of a 85% aqueous phosphoric acid solution and 82.16 parts of a 60% aqueous arsenic acid solution, and the mixture was heated and stirred at 92° C. for 10 hours to obtain a reddish brown transparent solution. Subsequently, 10.13 parts of antimony trioxide was added to the solution and heated and stirred for 4 hours to obtain a dark green transparent solution. Subsequently, the solution was spray-dried to obtain a dried slurry. The composition of the catalytically active component solid determined from the raw material charge amount is as follows:

Mo₁₀V_{0.5}P_{1.0}As_{0.5}Sb_{0.10}Cu_{0.2}O_{g}

(wherein g is a value determined by the valence and atomic ratio of other elements.)

Next, 214 parts of the resulting dried slurry and 29.8 parts of a strength improver (alumina-silica fiber) were uniformly mixed, and the mixture was coated and molded to 200 parts of a spherical porous alumina carrier (particle diameter: 3.8 mm) using about 30 parts of 90% aqueous ethanol solution as a binder. Next, the resulting molded product was subjected to calcination at 320° C. for 6 hours under air flow to obtain a catalyst (coated catalyst) of the invention.

### 2) Production of Methacrylic Acid

Into a stainless steel reaction tube having an inner diameter of 18.4 mm was filled 40.2 ml of the resulting coated catalyst of the invention, and a methacrolein oxidation reaction was performed under conditions of a raw material gas (composition (molar ratio); methacrolein:oxygen:water vapor:nitrogen=1:2:4:18.6) and space velocity (SV) 300 hr⁻¹. The reaction bath temperature was adjusted between 250° C. and 280° C., and the methacrylic acid selectivity when the methacrolein conversion rate was 77 mol % was calculated.

The conversion rate and selectivity are defined as follows.

Conversion rate=number of moles of methacrolein reacted/number of moles of methacrolein fed×100

Selectivity=number of moles of methacrylic acid produced/number of moles of methacrolein reacted×100

### 3) X-Ray Diffraction Measurement

X-ray diffraction measurement of the catalyst obtained was performed with the following apparatus and conditions.

Device used: (Ultima IV, manufactured by Rigaku Corporation)

### X-ray: CuKα ray (λ=0.154 nm)

### Output: 40 kV, 30 mA

Divergence slit: Open
Divergence vertical limit slit: 10 mm
Scattering slit; Open
Light receiving slit: 0.3 mm
Measurement range: 10 to 20°
Measurement speed: 10° per minute

### Step: 0.02°

Scan type: Continuous

### Example 2

A coated catalyst of the invention was prepared in the same manner as in Example 1 except that the calcining temperature of the molded catalyst in Example 1 was changed to 330° C.

### Example 3

A coated catalyst of the invention was prepared in the same manner as in Example 1 except that the calcining temperature of the molded catalyst in Example 1 was changed to 350° C.

### Example 4

A coated catalyst of the invention was prepared in the same manner as in Example 1 except that the calcining temperature of the molded catalyst in Example 1 was changed to 335° C.

### Example 5

A coated catalyst of the invention was prepared in the same manner as in Example 1 except that the calcining temperature of the molded catalyst in Example 1 was changed to 340° C.

### Example 6

A coated catalyst of the invention was prepared in the same manner as in Example 1 except that the calcining temperature of the molded catalyst in Example 1 was changed to 380° C.

### Example 7

To 7,100 parts of pure water were added 1,000 parts of molybdenum oxide, 37.91 parts of vanadium pentoxide, 11.05 parts of cupric oxide, 27.73 parts of copper acetate monohydrate, 96.09 parts of a 85% aqueous phosphoric acid solution and 82.16 parts of a 60% aqueous arsenic acid solution, and the mixture was heated and stirred at 92° C. for 10 hours to obtain a reddish brown transparent solution. Subsequently, 5.06 parts of antimony trioxide was added to the solution and heated and stirred for 4 hours to obtain a dark green transparent solution. Subsequently, the solution was spray-dried to obtain a dried slurry. The composition of the catalytically active component solid determined from the raw material charge amount is as follows:

Mo₁₀V_{0.6}P_{1.2}As_{0.5}Sb_{0.05}Cu_{0.4}O_{g}

(wherein g is a value determined by the valence and atomic ratio of other elements.)

Next, 214 parts of the resulting dried slurry and 29.8 parts of a strength improver (alumina-silica fiber) were uniformly mixed, and the mixture was coated and molded to 200 parts of a spherical porous alumina carrier (particle diameter: 3.8 mm) using about 30 parts of 90% aqueous ethanol solution as a binder. Next, the resulting molded product was subjected to calcination at 350° C. for 6 hours under air flow to obtain the desired coated catalyst.

### Example 8

To 7,100 parts of pure water were added 1,000 parts of molybdenum oxide, 44.23 parts of vanadium pentoxide, 11.05 parts of cupric oxide, 88.08 parts of a 85% aqueous phosphoric acid solution and 82.16 parts of a 60% aqueous arsenic acid solution, and the mixture was heated and stirred at 92° C. for 10 hours to obtain a reddish brown transparent solution. Subsequently, the solution was spray-dried to obtain a dried slurry. The composition of the catalytically active component solid determined from the raw material charge amount is as follows:

Mo₁₀V_{0.7}P_{1.1}As_{0.5}Cu_{0.2}O_{g}

(wherein g is a value determined by the valence and atomic ratio of other elements.)

Next, 214 parts of the resulting dried slurry and 29.8 parts of a strength improver (alumina-silica fiber) were uniformly mixed, and the mixture was coated and molded to 200 parts of a spherical porous alumina carrier (particle diameter: 3.8 mm) using about 40 parts of 90% aqueous ethanol solution as a binder. Next, the resulting molded product was subjected to calcination at 350° C. for 6 hours under air flow to obtain the desired coated catalyst.

### Comparative Example 1

A coated catalyst of the comparative example was prepared in the same manner as in Example 1 except that the calcining temperature of the molded catalyst in Example 1 was changed to 310° C.

### Comparative Example 2

### 1) Preparation of Catalyst

The dry slurry was prepared in the same manner as in Example 1. Next, 178 parts of the dried slurry and 24.8 parts of a strength improver (alumina-silica fiber) were uniformly mixed, and the mixture was coated and molded to 200 parts of a spherical porous alumina carrier (particle diameter: 3.8 mm) using about 25 parts of 90% by weight aqueous ethanol solution as a binder. Next, the resulting molded product was subjected to calcination at 310° C. for 5 hours under air flow to obtain a coated catalyst of the comparative example.

### Comparative Example 3

A coated catalyst of the comparative example was prepared in the same manner as in Example 1 except that the calcining temperature of the molded catalyst in Example 1 was changed to 400° C.

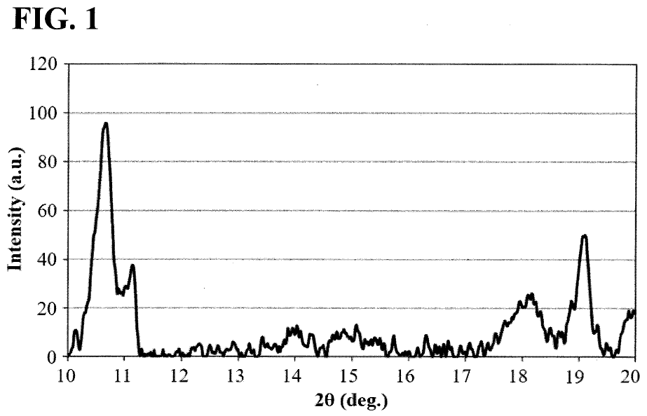

According to the same method as in Example 1, production of methacrylic acid using the catalysts of Examples 2 to 8 and Comparative Examples 1 to 3 and X-ray diffraction measurement of the catalyst were performed. These results are shown in Table 1 together with the results of the catalyst of Example 1. For reference, the X-ray diffraction spectrum obtained in the X-ray diffraction measurement of the catalyst of Example 1 is shown in FIG. 1.

TABLE 1

| | | (X-ray Diffraction | |
|---|---|---|---|
| | | Intensity of 19.1° ± | Methacrylic |
| | Calcining | 0.3°)/(X-ray | Acid |
| | Temperature | Diffraction Intensity | Selectivity |
| | (° C.) | of 10.7° ± 0.3°) | (mol %) |
| Example 1 | 320 | 0.52 | 82.8 |
| Example 2 | 330 | 0.48 | 86.1 |
| Example 3 | 350 | 0.44 | 86.9 |
| Example 4 | 335 | 0.41 | 85.3 |
| Example 5 | 340 | 0.30 | 86.6 |
| Example 6 | 380 | 0.20 | 86.2 |
| Example 7 | 350 | 0.33 | 84.3 |
| Example 8 | 350 | 0.28 | 85.7 |
| Comparative | 310 | 0.66 | 81.2 |
| Example 1 | | | |
| Comparative | 310 | 0.58 | 79.1 |
| Example 2 | | | |
| Comparative | 400 | 0.18 | 68.5 |
| Example 3 | | | |

As is apparent from Table 1, the catalysts of the invention in the examples have high methacrylic acid selectivity in comparison with the catalysts of the comparative examples.

While the invention has been described in detail with reference to specific embodiments, it will be apparent to those skilled in the art that various changes and modifications can be made without departing from the spirit and scope of the invention.

This application is based on a Japanese patent application filed on Mar. 28, 2018 (Japanese Patent Application No. 2018-62652), and the whole contents thereof are incorporated herein by reference. Also, all the references cited herein are incorporated as a whole.

### INDUSTRIAL APPLICABILITY

The present invention provides a catalyst for producing an unsaturated carboxylic acid represented by methacrylic acid in a high yield.

1. A catalyst for producing an unsaturated carboxylic acid,
wherein a ratio of a diffraction line intensity of 2θ=19.1±0.3° with respect to a diffraction line intensity of 2θ=10.7±0.3° in X-ray diffraction measurement is 0.20 or more and less than 0.58, and the catalyst comprising an active component represented by formula (A) shown below:
Mo₁₀VₐP_{b}Cu_{c}As_{d}XₑO_{g} (A)
in the formula, Mo, V, P, Cu, As and O represent molybdenum, vanadium, phosphorus, copper, arsenic and oxygen, respectively, and X represents at least one element selected from the group consisting of Ag, Mg, Zn, Al, B, Ge, Sn, Pb, Ti, Zr, Sb, Cr, Re, Bi, W, Fe, Co, Ni, Ce and Th; a, b, c, d, e and g each represents an atomic ratio of the element, and a satisfies 0.1≤a≤6, b satisfies 0.5≤b≤6, c satisfies 0<c≤3, d satisfies 0<d<3, e satisfies 0≤e≤3 and g is a value determined by the valence and atomic ratio of the other elements.

2. The catalyst according to claim 1,
wherein the unsaturated carboxylic acid is methacrylic acid.

3. A method for producing the catalyst according to claim 1 including the following steps:
(a) a step of mixing compounds containing an active component with water to prepare an aqueous solution or aqueous dispersion of these compounds,
(b) a step of drying the slurry solution obtained in the step (a) to obtain a dried slurry,
(c) a step of molding the dried slurry obtained in the step (b), and
(d) a step of calcining a molded product obtained in the step (c).

4. A method for producing an unsaturated carboxylic acid,
wherein an unsaturated aldehyde is gas phase catalytic oxidized by molecular oxygen using the catalyst according to claim 1.

5. The method according to claim 4,
wherein the unsaturated aldehyde is methacrolein and the unsaturated carboxylic acid is methacrylic acid.

6. A method for producing the catalyst according to claim 2 including the following steps:
(a) a step of mixing compounds containing an active component with water to prepare an aqueous solution or aqueous dispersion of these compounds,
(b) a step of drying the slurry solution obtained in the step (a) to obtain a dried slurry,
(c) a step of molding the dried slurry obtained in the step (b), and
(d) a step of calcining a molded product obtained in the step (c).

7. A method for producing an unsaturated carboxylic acid,
wherein an unsaturated aldehyde is gas phase catalytic oxidized by molecular oxygen using the catalyst according to claim 2.
